(19) United States
(12) Patent Application Publication
(10) Pub. No.: US 2026/0325870 A1
(43) Pub. Date: Oct. 22, 2026

(54) SYSTEMS AND METHODS FOR AI-DRIVEN LASER TREATMENT

(71) Applicant: Gary Goodman, LONGMEADOW, MA (US)

(72) Inventor: Gary Goodman, Longmeadow, MA (US)

(21) Appl. No.: 19/714,855

(22) Filed: Jun. 22, 2026

Related U.S. Application Data
(63) Continuation-in-part of application No. 19/459,380, filed on Jan. 26, 2026.
(60) Provisional application No. 63/833,942, filed on Jan. 29, 2025.

(51) Int. Cl. A61N 5/067; A61B 18/20; A61N 5/06

(52) U.S. Cl. CPC A61N 5/067; A61B 18/203; A61N 2005/0626; A61N 2005/0659; A61N 2005/0663

(57) ABSTRACT

A system for providing adaptive electromagnetic radiation therapy to a biological tissue of a subject comprises a plurality of electromagnetic radiation sources configured to emit electromagnetic radiation toward a treatment region of the subject; a plurality of physiological sensors configured to generate physiological data associated with a state of the treatment region; a controller operatively coupled to the electromagnetic radiation sources and the physiological sensors comprising one or more processors, a memory storing patient-specific data comprising at least one of historical treatment data for the subject, prior physiological response data or both; and a machine learning model executable by the controller and configured to process the physiological data and the patient-specific data to generate a control policy. The controller is configured, based on the control policy, to dynamically control at least one of: a selection of wavelengths emitted by the plurality of electromagnetic radiation sources, a waveform of the emitted electromagnetic radiation, a temporal sequence of activation of the plurality of electromagnetic radiation sources, and an intensity of the emitted electromagnetic radiation. The invention also provides methods for adaptive electromagnetic radiation therapy to the biological tissue of a subject.

This application is a continuation-in-part of U.S. patent application Ser. No. 19/459,380, entitled “METHODS AND DEVICES FOR WOUND HEALING,” filed Jan. 26, 2026, which claims priority to U.S. Provisional Patent Application No. 63/833,942, filed Jan. 29, 2025.

### TECHNICAL FIELD

The invention relates generally to laser treatment systems and more specifically to laser treatment systems and methods that incorporate artificial intelligence that include machine learning models for improved safety and enhanced outcomes.

### BACKGROUND

Photobiomodulation and low-level electromagnetic radiation therapy systems have been widely used for promoting biological tissue repair, modulating inflammatory responses, and supporting regenerative processes. Such systems typically deliver electromagnetic radiation, including visible or near-infrared light, to a target tissue region under predefined treatment parameters such as wavelength, intensity, and exposure duration.

WO2022234544A1 discloses a closed-loop photobiomodulation system configured to deliver optical stimulation based on physiological feedback signals. In this system, biometric or neurophysiological data acquired from a subject is processed to determine stimulation parameters, and optical stimulation is adaptively controlled in response to such feedback. Similarly, US20240238612A1 describes a system for adaptive light-based therapy in which physiological data is continuously acquired and used to modify stimulation patterns, including temporal modulation and intensity of emitted light, through algorithmic processing.

U.S. Ser. No. 12/263,352B2 discloses a photobiomodulation system for therapeutic treatment of biological tissue using electromagnetic radiation within specific wavelength ranges. The system applies optical energy to a target tissue region to induce therapeutic effects such as tissue repair or regeneration. However, the treatment parameters are generally defined according to predetermined protocols, with limited or no dynamic adaptation based on real-time physiological feedback.

U.S. Pat. No. 7,403,820B2 describes a closed-loop physiological control system in which one or more sensors detect physiological parameters of a subject and a controller adjusts stimulation signals in response thereto. The system provides feedback-based control of a therapeutic device; however, it is primarily directed to electrical or neural stimulation modalities rather than optical or multi-wavelength electromagnetic radiation systems.

U.S. Ser. No. 11/940,765B2 further discloses an intelligent therapeutic stimulation system that uses computational models, including rule-based or machine-learning-based approaches, to process physiological measurements and determine stimulation parameters. The system adapts therapeutic output in real time based on observed physiological conditions, but is not specifically directed to multi-wavelength photobiomodulation or coordinated optical emitter control.

Although the above systems disclose various forms of photobiomodulation, physiological feedback control, and algorithmic adaptation of therapeutic stimulation, several limitations remain. In particular, existing systems generally operate by adjusting individual parameters such as intensity or timing in isolation, or rely on predefined wavelength protocols without coordinated multi-parameter optimization.

Furthermore, known systems do not provide a unified control architecture capable of simultaneously coordinating multiple electromagnetic radiation parameters, including wavelength selection, waveform modulation, emitter sequencing, and intensity control, in a dynamically adaptive manner based on integrated multi-source data. In addition, prior systems typically do not integrate longitudinal patient-specific treatment histories, population-level datasets, and real-time physiological feedback within a single unified control framework for generating adaptive treatment policies.

Accordingly, there exists a need for an improved system and method for electromagnetic radiation therapy that is capable of dynamically optimizing multiple treatment parameters in real time. In particular, there is a need for a system that integrates multi-source data, including historical patient data, population-level knowledge, and real-time physiological feedback, to generate adaptive control policies for coordinated multi-wavelength electromagnetic radiation delivery.

There is further a need for a system capable of controlling multiple electromagnetic radiation emitters in a coordinated manner, including simultaneous, sequential, and hybrid emission strategies, while dynamically adjusting waveform characteristics and intensity based on evolving physiological conditions of a subject during treatment.

### BRIEF DESCRIPTION

The present invention addresses these needs by providing a system and method for adaptive electromagnetic radiation therapy in which a machine learning model processes multi-layer data inputs and generates control outputs for a controller. The controller is configured to regulate a plurality of electromagnetic radiation emitters, including control over wavelength selection, waveform modulation, emission sequencing, and intensity, in response to real-time physiological feedback and stored patient-specific data.

In one aspect, the invention provides a system for providing adaptive electromagnetic radiation therapy to a biological tissue of a subject. The system comprises a plurality of electromagnetic radiation sources configured to emit electromagnetic radiation toward a treatment region of the subject. The system also comprises a plurality of physiological sensors configured to generate physiological data associated with a state of the treatment region. A controller comprising one or more processors is provided as part of the system, wherein the controller is operatively coupled to the electromagnetic radiation sources and the physiological sensors. Further, a memory storing patient-specific data comprising at least one of historical treatment data for the subject, prior physiological response data, or both is also made available as part of the system. The system then comprises a machine learning model executable by the controller and configured to process the physiological data and the patient-specific data to generate a control policy. The controller is configured, based on the control policy, to dynamically control at least one of: a selection of wavelengths emitted by the plurality of electromagnetic radiation sources, a waveform of the emitted electromagnetic radiation, a temporal sequence of activation of the plurality of electromagnetic radiation sources, and an intensity of the emitted electromagnetic radiation.

The system is configured to operate in a closed-loop manner such that the control policy is iteratively updated during a treatment session based on feedback derived from the physiological data, thereby adapting delivery of electromagnetic radiation to the biological tissue.

In another aspect, the invention provides a method for providing adaptive electromagnetic radiation therapy to a biological tissue of a subject. The method comprises emitting electromagnetic radiation from a plurality of electromagnetic radiation sources toward a treatment region of the subject, followed by acquiring physiological data associated with a state of the treatment region using a plurality of physiological sensors. The method then involves processing the physiological data and patient-specific data using a machine learning model to generate a control policy that is iteratively updated during a treatment session based on feedback derived from the physiological data. Subsequently, the method comprises dynamically controlling at least one of wavelength selection, waveform, temporal sequencing, or intensity of the electromagnetic radiation based on the control policy. In this manner, electromagnetic radiation delivery to the biological tissue is adaptively regulated during any treatment session that results in greater safety for the patient and/or improved outcomes.

### BRIEF DESCRIPTION OF DRAWINGS

These and other features, aspects, and advantages of the present invention will become better understood when the following detailed description is read with reference to the accompanying drawings in which like reference numerals represent corresponding parts throughout the drawings, wherein:

FIG. 1 is a block diagram representation of exemplary components comprising the system of the invention.

FIG. 2 is a flowchart representation of exemplary steps involved in the method of the invention.

### DETAILED DESCRIPTION OF THE INVENTION

The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the term “or” is generally employed in its sense including “and/or” unless the content clearly dictates otherwise.

As used herein, the term “electromagnetic radiation source”, also sometimes abbreviated as “EMR”, refers to an element of the electromagnetic radiation therapy apparatus that is configured to provide an optical output (e.g., to transmit light from a light therapy apparatus to a target tissue, such as an ocular tissue, of a patient). These may include any type of light sources, which comprises, for example, but not limited to, light emitting diodes (LEDs), laser diodes, intense pulsed light, and the like, and combinations thereof. The emitted electromagnetic radiation is characterized by a number of parameters, such as wavelength, intensity, amplitude, and the like. In some embodiments, the electromagnetic radiation has a specific wavelength, while in other embodiments, the electromagnetic radiation has a band of wavelengths.

As used herein, the term “biological tissue” refers to any aggregation of biological cells and/or extracellular components forming a structural and/or functional unit of a living organism. Biological tissue may include, without limitation, epithelial tissue, connective tissue, muscle tissue, neural tissue, vascular tissue, dermal tissue, subdermal tissue, mucosal tissue, or any combination thereof. The term further encompasses normal, injured, diseased, inflamed, ischemic, or regenerating tissue, as well as tissue undergoing repair, remodeling, or other physiological or pathological processes. Biological tissue may be located at or within any anatomical region of a subject, including skin, subcutaneous layers, organs, musculoskeletal structures, neural structures, or vascular systems. The term is intended to include both superficial and deep tissue structures, and may further include cellular microenvironments, tissue interfaces, and regions of tissue adjacent to a target treatment area that are affected by or responsive to applied electromagnetic radiation. In some embodiments, biological tissue includes wound tissue, scar tissue, burn-affected tissue, or tissue associated with impaired healing. In other embodiments, biological tissue includes healthy tissue that is subjected to electromagnetic radiation for therapeutic, modulatory, or prophylactic purposes. The term is intended to be broadly construed and is not limited to any specific tissue type, anatomical location, or physiological condition, provided that the tissue is capable of interacting with electromagnetic radiation and exhibiting a measurable physiological response.

As used herein, the term “physiological data” refers to data indicative of one or more states, conditions, or responses of a biological tissue or subject. Physiological data may include baseline physiological state data, real-time physiological response data, or combinations thereof. Such data may be derived from one or more physiological sensors and may include, without limitation, measurements of temperature, oxygenation, perfusion, blood flow, optical absorption, electrical activity, metabolic activity, or other biologically relevant parameters indicative of tissue state or response to electromagnetic radiation.

As used herein, the term “baseline physiological state data” refers to physiological data acquired prior to initiation of a treatment session involving electromagnetic radiation. Baseline physiological state data characterizes an initial condition of a biological tissue or treatment region prior to exposure to electromagnetic radiation and may be used to initialize or parameterize a control policy.

As used herein, the term “real-time physiological response data” refers to physiological data acquired during application of electromagnetic radiation therapy. Such data reflects dynamic changes in a biological tissue or treatment region in response to delivered electromagnetic radiation and may be used to iteratively update a control policy during a treatment session.

As used herein, the term “physiological sensor” refers to any device, system, or component capable of detecting, measuring, or generating data indicative of a physiological state or response of a biological tissue or subject. Physiological sensors may include optical sensors, thermal sensors, photodetectors, spectroscopic sensors, impedance sensors, or combinations thereof.

As used herein, the term “controller” refers to a hardware-based system comprising one or more processors configured to execute instructions stored in memory. A controller may include digital processors, microcontrollers, or processing units and may be configured to perform signal processing, control logic execution, and generation of control signals for regulating electromagnetic radiation sources.

As used herein, the term “machine learning model” refers to a computational model configured to generate outputs based on learned relationships derived from training data. The term “machine learning” may sometimes be abbreviated as “ML”. The machine learning model may include, without limitation, neural networks, reinforcement learning models, regression models, decision trees, or hybrid models. In various embodiments, the machine learning model is configured to process physiological data and patient-specific data to generate a control policy for controlling electromagnetic radiation therapy parameters.

As used herein, the term “control policy” refers to a set of computed instructions, parameters, or rules generated by a machine learning model or controller that defines how electromagnetic radiation sources are to be operated during a treatment session. A control policy may define one or more of wavelength selection, waveform characteristics, temporal sequencing, and intensity levels, and may be updated dynamically during a treatment session based on physiological feedback.

As used herein, the term “patient-specific data” refers to data associated with a specific subject receiving electromagnetic radiation therapy. Patient-specific data may include historical treatment data, prior physiological response data, longitudinal treatment records, demographic data, anatomical characteristics, or combinations thereof.

As used herein, the term “historical treatment data” refers to data generated from one or more prior treatment sessions involving electromagnetic radiation therapy. Such data may include treatment parameters, emitted wavelengths, waveform characteristics, intensity levels, duration of exposure, and associated physiological outcomes.

As used herein, the term “control policy update” refers to the modification of a control policy during a treatment session based on newly acquired physiological data. A control policy update may occur iteratively and in real time, such that electromagnetic radiation parameters are dynamically adapted in response to changes in tissue state.

As used herein, the term “temporal sequencing” refers to the ordered activation of two or more electromagnetic radiation sources over time. Temporal sequencing may include sequential, simultaneous, or hybrid activation patterns, and may be dynamically determined based on a control policy.

As used herein, the term “waveform” refers to the temporal profile of electromagnetic radiation emission, including variations in amplitude, frequency, duty cycle, or modulation pattern. Waveforms may include sinusoidal, square, pulsed, or other time-varying emission profiles.

As used herein, the term “closed-loop system” refers to a system in which output signals, including physiological response data, are continuously or periodically fed back into a controller or machine learning model to adjust subsequent electromagnetic radiation delivery parameters during a treatment session.

As used herein, the term “treatment region” refers to a portion of biological tissue of a subject to which electromagnetic radiation is applied. The treatment region may include a wound site, lesion, anatomical structure, or any target tissue area selected for therapy.

As used herein, the term “adaptive electromagnetic radiation therapy” refers to a therapeutic process in which electromagnetic radiation parameters are dynamically adjusted during a treatment session based on physiological data, patient-specific data, and control policies generated by a machine learning model or controller.

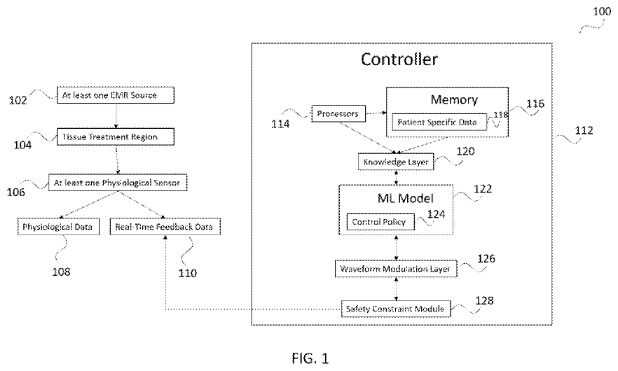

As noted herein, in one aspect the invention provides a system for providing adaptive electromagnetic radiation therapy to a biological tissue of a subject. FIG. 1 is a block diagrammatic representation of exemplary components belonging to the system of the invention depicted by numeral 100. The system 100 comprises at least one electromagnetic radiation source 102 configured to emit electromagnetic radiation toward a tissue treatment region 104 of the subject. The at least one electromagnetic radiation source includes at least a first electromagnetic radiation source configured to emit a first wavelength and a second electromagnetic radiation source configured to emit a second wavelength different from the first wavelength. In some embodiments, the first wavelength ranges from about 800 nm to about 1300 nm and the second wavelength ranges from about 400 nm to about 500 nm. In some specific embodiments, the second electromagnetic radiation source is configured to emit light at about 450 nm.

In some additional embodiments, the at least one electromagnetic radiation source comprises a third electromagnetic radiation source configured to emit electromagnetic radiation having a wavelength ranging from about 600 nm to about 700 nm.

The system 100 also comprises at least one physiological sensor 106 configured to generate physiological data 108 indicative of a response of the treatment region to the emitted electromagnetic radiation. The physiological data includes at least one of, but not limited to, temperature, blood perfusion, oxygen saturation, optical response signals, or combinations thereof. The instantaneously generated physiological data is a real-time feedback data 110 that will be used for controlling the therapy session as described herein.

The system 100 then comprises a controller 112 comprising one or more processors 114, wherein the controller is operatively coupled to the electromagnetic radiation sources and the physiological sensors. The system further includes a memory 116 for storing patient-specific data 118 comprising at least one of historical treatment data for the subject and prior physiological response data. The stored patient-specific treatment data 118 may comprise, for example, prior electromagnetic radiation exposure parameters, corresponding physiological response data, and the like, and combinations thereof.

A knowledge layer 120 is also included that stores data from external sources, such as, but not limited to, clinical data, information from relevant literature sources, clinical trials data, and so on. The knowledge layer 120 is also configured to additionally analyze the data and make it more usable in the system of the invention, which may include one or more steps of, curating the data, adding metadata, tagging with appropriate codes, categorizing, summarizing, and the like, and combinations thereof. The knowledge layer 120 may further assimilate past treatment data to augment the existing data.

The system 100 comprises a machine learning model 122 executable by the controller 110 and configured to process (i) the physiological data 108, (ii) the patient-specific data 118, and (iii) the real-time feedback data 110 from the at least one physiological sensor to generate a control policy 124. The controller 112 is configured, based on the control policy 124, to dynamically control at least one of: wavelength selection of the electromagnetic radiation sources, waveform of the electromagnetic radiation, temporal sequencing of activation of the electromagnetic radiation sources, intensity of the electromagnetic radiation, or combinations thereof, wherein the control policy 124 is updated during a treatment session based on continuous physiological feedback from the real-time feedback data 110, thereby providing a closed-loop adaptive electromagnetic radiation therapy system. The machine learning model 122 is updated across multiple treatment sessions based on longitudinal patient response data. The controller further accesses population-based treatment data used to train or initialize the machine learning model and from all other users across the learning model.

In one embodiment, the system of the invention is configured such that the electromagnetic radiation sources are configured to operate in a simultaneous emission mode. In other embodiments, the system of the invention is configured such that the electromagnetic radiation sources are configured to operate in a sequential emission mode determined by the control policy. In yet other embodiments, the system of the invention is configured such that the electromagnetic radiation sources are configured to operate in a hybrid mode comprising both simultaneous and sequential emission within a single treatment session. The operation of the electromagnetic radiation sources is effected by suitable programming made available from the memory 116 and executed by the one or more processors 114.

The controller 112 comprises a waveform modulation layer 126 configured to generate at least one of a sinusoidal waveform, square waveform, or pulsed waveform and may further comprise a multi-wavelength coordination engine configured to determine a temporal order of activation of the electromagnetic radiation sources. The machine learning model 122 is trained to map physiological signals and stored treatment data to emitter control parameters that is then fed to the controller 112, which in turn is used to control a specific treatment session. In some embodiments, the controller 112 is configured to modify the electromagnetic radiation output during a treatment session in response to changes in the real-time physiological feedback data 110. In another exemplary embodiment, the controller 112 is configured to adjust relative intensity among the first and second electromagnetic radiation based on real-time physiological feedback data 110. Thus, the controller 112 performs iterative adjustment of electromagnetic radiation output during a single treatment session based on continuously updated physiological feedback.

The controller 112 further comprises a safety constraint module 128 to ensure the entire therapy session is conducted within the safety parameters so as to prevent any accidents and/or injuries. The safety constraint module 128 is configured to limit at least one of, but not limited to, exposure duration, energy density, or temperature increase of the biological tissue. Some exemplary constraints may include, for example, but not limited to, the energy density of the electromagnetic radiation during a treatment session ranges from about 1 mJ/cm² to about 1000 J/cm², the power of the electromagnetic radiation does not exceed 1000 W, the exposure time of the biological tissue to the electromagnetic radiation at any given time does not exceed 100 minutes continuously, and the like, and combinations thereof.

The machine learning model 122 described herein is further configured to update the control policy during the treatment session based on continuous physiological feedback.

The stored patient-specific treatment data is updated after each treatment session based on measured outcomes. The controller 112 is configured to assign different weighting to stored treatment data and real-time physiological feedback data 110 when generating the control output, the exact weighting may depend on various factors, such as, but not limited to, patient details, medical history, concurrent treatment, current symptoms, prior responses to treatment, external factors such as other patients' responses, potential side effects from peer reviewed studies, and the like, and combinations thereof.

In some instances, the electromagnetic radiation is configured to perform tissue debridement. The debridement may be preliminary step to a treatment session, or alternately, it may be performed in the middle of a treatment session, which may or may not be performed as a result of feedback from the one or more physiological sensor. Suitable wavelengths for tissue debridement may include wavelengths from about 1400 nm to about 1500 nm, specifically 1470 nm. The 1400 nm-1500 nm wavelength electromagnetic radiation may further be advantageously used in combination with a 980 nm electromagnetic radiation. Without being bound to theory, it is known that a combination of 980 and 1470 nm lasers results in a balance of tissue penetration and thermal effect. The 980 nm wavelength is effective for cutting and coagulation, while the 1470 nm wavelength is better for promoting collagen regeneration and skin texture improvement. This dual-wavelength approach allows for a more versatile and effective treatment, and can improve procedures such as tissue cutting, cropping off necrotic tissue, tissue debridement, and the like. The combination of these wavelengths also provides a safer and more controlled treatment experience, reducing the risk of side effects and improving patient outcomes.

Thus, the system of the invention 100 described herein is used in a therapeutic system configured for, for example, but not limited to, wound healing, pain management, dermatological treatment, deep tissue healing, hair growth stimulation, musculoskeletal therapy, tissue regeneration, and the like, and combinations thereof. The system of the invention is configured to take into account advances in the field by studying and analyzing published peer-reviewed studies to update the machine learning model. Then, all past historical treatment data is also appropriately tagged and updated in the machine learning model. These training data are then combined appropriately in the ML model to make it a strong model. Subsequently, when this is combined with a patient specific data, a robust treatment plan can be arrived at that will ensure favorable outcomes from every treatment session.

In one exemplary embodiment, a peer-reviewed publication that concludes that 980 nm laser provides better healing abilities but results in poor thermal characteristics in the region when compared with a 810 nm is fed into the machine learning model. This study is analyzed and tagged with certain specific characteristics from the study (e.g. oral wound healing or skin incision). The ML model is updated with this study and then existing historical treatment data is then scoured by the model to identify all the treatment sessions to extract data that concurs with and/or counters this conclusion. The model is then updated to include this information for future sessions, while any data points that counter this would be analyzed deeper to offer any possible explanations. If there are no historical data points available for this, then all future treatment sessions would be modified to obtain this data also using the appropriate physiological sensors (e.g. use a thermal scanner to obtain thermal profile of the region being exposed). Then, for a particular treatment session, this information from the model would be utilized to arrive at a treatment plan. For example, for an internal wound such as inside a patient's mouth, 810 nm may be preferred as the thermal characteristics are better, whereas for a skin wound, a 980 nm may be preferred as temperature tolerance may be greater.

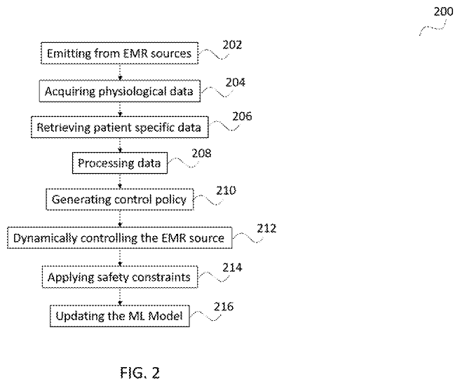

In another aspect, the invention provides a method for providing adaptive electromagnetic radiation therapy to a biological tissue of a subject. FIG. 2 is a flowchart representation of the method of the invention represented by numeral 200 showing exemplary steps therein. The method 200 comprises emitting electromagnetic radiation 202 from at least one electromagnetic radiation source toward a treatment region of the subject. The at least one electromagnetic radiation source is configured to provide electromagnetic radiation having at least two different wavelengths. One skilled in the art will understand that a single electromagnetic radiation source can be used to generate electromagnetic radiation having more than one wavelength, or multiple electromagnetic radiation sources may be used to generate electromagnetic radiations of different wavelengths.

The method 200 then comprises acquiring physiological data in step 204 from the treatment region using at least one physiological sensor. Subsequently, patient-specific data comprising at least one of historical treatment data or prior physiological response data is retrieved in step 206. The method then involves processing in step 208 the physiological data, the patient-specific data, and real-time feedback data using a machine learning model to generate a control policy in step 210 that comprises processing multi-modal data using the machine learning model to determine optimized treatment parameters. Further, the control policy is updated in real time during the treatment session based on physiological feedback.

As a result of application of control policy, the method then involves dynamically controlling 212 the electromagnetic radiation source at least one of wavelength selection, waveform, temporal sequencing, or intensity of the electromagnetic radiation based on the control policy. The method is configured, in some embodiments, to be updated during a treatment session based on continuous physiological feedback, thereby forming a closed-loop adaptive electromagnetic radiation therapy method.

The method of the invention further comprises applying safety constraints in step 214 to limit at least one of exposure energy, intensity, or temperature of the biological tissue. An exemplary safety constraint may include the electromagnetic radiation to be delivered with an energy density ranging from about 1 mJ/cm² to about 1000 J/cm². Another exemplary safety constraint may be that the power of the electromagnetic radiation source does not exceed 1000 W.

As already noted, emitting electromagnetic radiation comprises simultaneously emitting electromagnetic radiation from at least two electromagnetic radiation sources, sequentially activating the plurality of electromagnetic radiation sources according to the control policy, or a combination of simultaneous and sequential emission during a single treatment session.

Also as already noted, modulating a waveform of the electromagnetic radiation may include at least one of a sinusoidal waveform, square waveform, pulsed waveform, or combinations thereof.

The method also involves updating the machine learning model in step 216 using inputs from across multiple treatment sessions based on longitudinal patient response data. Further, the stored patient-specific treatment data is updated after each treatment session based on measured outcomes.

As can be gleaned, the method of the invention 200 is advantageously applied, for example, but not limited to, for wound healing, pain alleviation, deep tissue healing, hair regrowth, tissue regeneration, and the like, and combinations thereof.

It will be appreciated, in view of the present disclosure, embodiments of the present disclosure may be implemented as or include one or more general-purpose or special purpose computing systems, which may take on a variety of forms that may include, besides one or more of the components already mentioned herein, I/O system(s), communication system(s), and/or additional or alternative components. The memory unit includes a database having pre-stored data, wherein the pre-stored data may include, besides those data points already mentioned herein, for example a plurality of user profiles corresponding to a plurality of respective users. The database in the memory unit may also include therapeutic programs associated with body parts, skin types, and combinations thereof. This would enable calling up of a suitable therapeutic program by the device based on, for example, an initial or instant image obtained from the camera, or from inputs related to an initial condition of a biological tissue.

The processor(s) may comprise one or more sets of electronic circuitries that include any number of logic units, registers, and/or control units to facilitate the execution of computer-readable instructions (e.g., instructions that form a computer program). At least a portion of processing of instructions is performed by ICs on a PCB. The ICs may also comprise at least a portion of the memory comprising the instructions to be executed by the processor. Such computer-readable instructions may be stored within the memory unit. The memory unit may comprise physical system memory and may be volatile, non-volatile, or some combination thereof. Furthermore, memory unit may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. For example, processor(s) may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, facilitating the disclosed embodiments may rely at least in part on communication system(s) for receiving data from or facilitating coordinated execution with remote system(s). Remote systems may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others. In further embodiments, communications system(s) may comprise a Subscriber Identity Module (SIM) card that may be used to connect to mobile networks, satellite networks, or combinations thereof, which allows for connectivity even in remote areas that resource stressed and/or lacking proper infrastructure.

Furthermore, in some instances, facilitating the disclosed embodiments may rely at least in part on I/O system(s). I/O system(s) may include any type of input or output device such as, by way of non-limiting example, a touch screen, a mouse, a keyboard, and/or others, without limitation.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RANI, ROM, EEPROM, CD-ROM, solid state drives (“SSD”) that are based on RANI, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer. Storage media made available remote from the system, such as a “cloud” server is also envisioned to be included as storage media in the invention.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RANI within a network interface module (e.g., a “NIC”), and then eventually transferred to computer system RANI and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory unit devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

### EXAMPLES

### Example 1

### Independent Evidence for Diode Laser Tissue Cutting

### Summarized Studies at 450 nm, 810 nm, and 980 nm Wavelengths

Inputs for training the machine learning model may include information made available from publications of peer reviewed studies. In this example, peer reviewed publications were analyzed, summarized, tagged and added to the machine learning model such that it is called upon for when such a procedure is implemented. Specifically, given below is a compilation of published, peer-reviewed studies demonstrating the tissue-cutting efficacy of three diode laser wavelengths—450 nm (blue), 810 nm (near-IR), and 980 nm (near-IR)—across multiple tissue types and clinical settings. Studies are organized by wavelength with key parameters and outcomes summarized for each.

450 nm—Blue Diode Laser

### 1. Oral Soft Tissue Excision (In Vivo)

Fornaini C, Rocca J P, Merigo E. “450 nm diode laser: A new help in oral surgery.” World J Clin Cases. 2016; 4(9):253-257.
Procedure: Lower lip fibroma removal using 450 nm blue diode laser
Parameters: 1 W continuous wave; topical anesthesia only
No suture required; healing completed in one week
Histology: Regular incision line with very scanty areas of carbonization
Patient reported no pain, discomfort, or need for analgesics post-operatively
Key Finding: High surgical efficiency at very low power (1 W), with minimal thermal damage and excellent patient comfort.

### 2. Bladder Tissue Vaporization (Ex Vivo/In Vivo)

Jiang D L, Yang Z, Liu G X, et al. “A novel 450-nm blue laser system for surgical applications: efficacy of specific laser-tissue interactions in bladder soft tissue.” Lasers Med Sci. 2019; 34(4):807-813.
Compared 30 W blue (450 nm) laser vs. 532 nm green laser on porcine bladder tissue
Vaporization efficiency: blue laser 5.14 mm³/s vs. green laser 1.20 mm³/s (4.3×advantage)
Coagulation depth: blue 460±70 microns vs. green 470±80 microns (comparable hemostasis)
In vivo canine transurethral resection simulation confirmed equivalent safety and efficacy
Low thermal damage to adjacent tissues; low laser-tissue penetration depth
Key Finding: 450 nm achieves 4.3× greater vaporization efficiency than the clinical standard (532 nm green laser) with equivalent coagulation and minimal collateral damage.
   810 nm—Near-Infrared Diode Laser

### 1. Breast Mass Excision (Ex Vivo/In Vivo)

Mahmood A S. “Evaluation of breast mass excision by 810 nm diode laser.” Iraqi J Laser. 2007; 6(1):31-35.
810 nm diode laser with 600 mm fiber, conical tip; 15 W (5.3 kW/cm² power density)
Three female patients (mean age 35.5 y) with clinically palpable breast masses
Cutting depth: 2-3 mm; cutting speed: 0.6 cm/s
Overall blood loss significantly less than conventional scalpel method
Clear surgical margins; hemostatic capability ensured anatomical visualization
No wound infection; reduced postoperative pain, swelling, and edema
Key Finding: 810 nm diode laser is effective for breast mass excision with superior hemostasis, clear margins, and reduced post-operative morbidity vs. conventional technique.

### 2. Oral Soft Tissue Surgery (In Vivo)

Akbulut N, Kursun E S, Tumer M K, et al. “Is the 810-nm diode laser the best choice in oral soft tissue therapy?” Eur J Dent. 2013; 7(2):207-211.
27 patients treated for benign oral lesions (frenulum, epulis fissuratum, lichen planus, leukoplakia, pyogenic granuloma)
Simple soft tissue lesions healed within 2 weeks; pyogenic granuloma within 4 weeks
White and vesiculobullous lesions healed completely within 6 weeks
No complications reported with 810 nm diode laser treatment
High degree of patient acceptance and satisfaction
Key Finding: 810 nm diode laser offers reliable, complication-free excision across a broad range of oral soft tissue pathologies with high patient satisfaction.
   980 nm—Near-Infrared Diode Laser

### 1. Oral Lesion Excision Comparative Study (In Vivo)

Tawi S R, Ahmed S S, Algarak B S. “Histopathological evaluation of two diode lasers 450 and 980 nm in excision biopsies of oral benign lesions.” Lasers Dent Sci. 2023; 7:251-258.
38 patients randomized to 450 nm or 980 nm excision groups
Both wavelengths: 1.3 W pulsed mode, 400 microns tip diameter
980 nm demonstrated higher cutting efficiency than 450 nm
980 nm produced a higher thermal effect and less precise cutting line
450 nm yielded clearer, more easily readable histological specimens
Key Finding: 980 nm cuts faster but with greater thermal effect; 450 nm provides superior histological clarity and precision. Both are clinically effective.

### 2. Fibroma Removal (In Vivo)

La Terra S L, Caccianiga G, Buoncristiani F, et al. “Clinical Outcomes of Oral Traumatic Fibroma Removal Using a 980 nm Diode Laser: A Series of Four Cases.” Cureus. 2025; 17(3):e80390.
Four patients (ages 35-78); lesions 0.8-1.5 cm
Parameters: 2 W continuous wave, contact mode
Minimal bleeding; complete fibroma removal in all cases
Uneventful healing within 7-9 days; no recurrence at 6 months
Mild postoperative pain; high patient satisfaction scores
Key Finding: 980 nm diode laser at low power (2 W) provides safe, effective fibroma excision with rapid healing and no recurrence.

### 3. Colon Tissue Cutting (Ex Vivo/In Vitro)

Ren Z H, Chai K, Zhao C, et al. “Dual-wavelength (980/1470 nm) laser incisions in porcine colon tissue: a histological in vitro study.” Lasers Med Sci. 2025; 40:253.
980 nm laser tested on fresh porcine colon tissue at 2 mm/s traverse speed
Complete colon wall penetration (mucosa through serosa) achieved at 72 W with 980 nm alone
Compared to 1470 nm (50 W) and dual-wavelength 980/1470 nm (6:6 W total)
980 nm efficiently absorbs both hemoglobin and water chromophores
Single-wavelength 980 nm showed higher thermal damage than dual-wavelength approach
Key Finding: 980 nm alone achieves full-thickness colon wall cutting but requires high power (72 W); pairing with 1470 nm dramatically reduces the power threshold while preserving hemostatic benefit.

Comparison of Cutting Characteristics by Wavelength

| | | | |
|---|---|---|---|
| Parameter | 450 nm (Blue) | 810 nm (Near-IR) | 980 nm (Near-IR) |
| Primary | Hemoglobin (peak | Melanin, | Water, hemoglobin |
| Chromophore | absorption) | hemoglobin | |
| Typical Cutting | 1-30 W | 2-15 W | 2-72 W |
| Power | | | |
| Cut Precision | Highest | Moderate | Moderate |
| Thermal Damage | Minimal | Low-Moderate | Moderate-High |
| Zone | | | |
| Carbonization | Very scanty | Low | Higher |
| Hemostatic | Good | Excellent | Excellent |
| Capability | | | |
| Coagulation Depth | 460 ± 70 mm* | Effective to 2-3 mm | Higher than 450 nm; |
| | | cutting depth | deep penetration |
| Typical Healing | ~1 week | 2-6 weeks (lesion- | 7-9 days |
| (Oral Soft Tissue) | | dependent) | |
| Histological | Best readability; | Good diagnostic | Lower readability; |
| Specimen Quality | clear margins | quality | more artifact |
| Key Advantage | Precision at very low | Versatile tissue | Strong hemostasis; |
| | power | interaction; | high cutting |
| | | hemostasis | efficiency |
| Key Limitation | Limited deep-tissue | Moderate thermal | Greater thermal |
| | penetration data | spread | damage; requires |
| | | | higher power for |
| | | | thick tissue |
| Tissue Types | Oral mucosa, bladder | Breast tissue, oral | Oral mucosa, colon |
| Studied | epithelium | mucosa | wall |

*Coagulation depth for 450 nm measured at 30 W on porcine bladder tissue (Jiang et al., 2019).

All studies cited are published in peer-reviewed journals. This compilation is intended as a reference summary for evaluating diode laser wavelength suitability across tissue types. Individual study parameters, sample sizes, and methodologies vary. Clinical decision-making should consider the full body of evidence and manufacturer specifications.

### Example 2

### Photobiomodulation Safe Dosimetry Summary

This example provides a method of calculation of certain treatment parameters during a photobiomodulation treatment session.

### Photobiomodulation—Maximum Wattage & Fastest Treatment Time

Input Parameters Parameter Value
Wound Diameter 4 mm (0.4 cm)
Target dose (fluence) 10 J/cm2
Maximum safe PBM Irradiance 0.1 W/cm2

### Wound Area Calculation

For a circular wound the area is computed as:

A=π×r²=p×(0.2 cm)²=π×0.0400 cm²

### A=0.1257 cm²

### Maximum Safe Wattage

The maximum optical power that keeps irradiance at or below the safety limit:

P_max=Irradiance_max×A=0.1 W/cm²×0.1257 cm²

P_max=0.01257 W>>12.57 mW

### Fastest Safe Treatment Time

Total energy required and the minimum treatment duration at maximum safe power:

E_total=Dose×A=10 J/cm²×0.1257 cm²=1.2566 J

t_min=E_total/P_max=1.2566 J/0.01257 W

t_min=100.0 seconds (1.67 min)

### Results at a Glance

Wound Area 0.1257 cm²
Max Safe Wattage 0.01257 W (~12.6 mW)
Fastest Safe Time 100 seconds (1.67 min)

In the example given herein, treatment session plan is generated based on the input parameters. Further, control policy that includes safety constraints are also established mathematically to ensure subject is not exposed to high power radiation and/or exposed for too long causing potentially irreparable damage to tissue.

### Example 3

This example illustrates the calculation of treatment given a certain set of parameters during a treatment session. For a 0.2 W Laser, the calculation is as follows:
Power=0.2 W
Energy=4 J
Calculated time=20 seconds
   The calculation is based on the formula:

[ext{Time}=rac{ext{Energy}}{ext{Power}}]

### Where:

Power is the laser power in Watts
Energy is the total energy delivered in Joules
For this example,

[ext{Time}=rac{ext{4,J}}{ext{0.2.W}}=20,s].

This means the laser has to be applied for 20 seconds to deliver 4 Joules of energy at 0.2 Watts power.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed:

1. A system for providing adaptive electromagnetic radiation therapy to a biological tissue of a subject, the system comprising:
a plurality of electromagnetic radiation sources configured to emit electromagnetic radiation toward a treatment region of the subject, the electromagnetic radiation sources including at least a first electromagnetic radiation source configured to emit a first wavelength and a second electromagnetic radiation source configured to emit a second wavelength different from the first wavelength;
a plurality of physiological sensors configured to generate physiological data associated with a state of the treatment region;
a controller comprising one or more processors, the controller being operatively coupled to the electromagnetic radiation sources and the physiological sensors;
a memory storing patient-specific data comprising at least one of historical treatment data for the subject or prior physiological response data; and
a machine learning model executable by the controller and configured to process the physiological data and the patient-specific data to generate a control policy;
wherein the controller is configured, based on the control policy, to dynamically control at least one of:
a selection of wavelengths emitted by the plurality of electromagnetic radiation sources,
a waveform of the emitted electromagnetic radiation,
a temporal sequence of activation of the plurality of electromagnetic radiation sources, and
an intensity of the emitted electromagnetic radiation,
wherein the control policy is iteratively updated during a treatment session based on feedback derived from the physiological data, thereby adapting delivery of electromagnetic radiation to the biological tissue.

2. The system of claim 1, wherein the physiological data includes data indicative of at least one of: temperature, microbial population information, of the treatment region prior to and during application of electromagnetic radiation.

3. The system of claim 1, wherein the physiological sensors are further configured to acquire baseline physiological state data of the treatment region prior to initiation of electromagnetic radiation therapy.

4. The system of claim 1, wherein the physiological sensors are further configured to generate real-time physiological response data during application of electromagnetic radiation.

5. The system of claim 1, wherein the physiological data comprises both baseline physiological state data and real-time physiological response data such that the control policy is based on a temporal evolution of the treatment region.

6. The system of claim 1, further comprising a third electromagnetic radiation source configured to emit electromagnetic radiation having a wavelength different from the first and second wavelengths.

7. The system of claim 1, wherein the first wavelength ranges from about 800 nm to about 1300 nm and the second wavelength ranges from about 400 nm to about 500 nm.

8. The system of claim 7, wherein first electromagnetic radiation source is configured to emit electromagnetic radiation at about 980 nm and the second electromagnetic radiation source is configured to emit electromagnetic radiation at about 450 nm.

9. The system of claim 1, wherein the controller is configured to activate the plurality of electromagnetic radiation sources in a sequence determined by the control policy.

10. The system of claim 1, wherein the plurality of electromagnetic radiation sources are configured to emit electromagnetic radiation simultaneously.

11. The system of claim 1, wherein the plurality of electromagnetic radiation sources are configured to emit electromagnetic radiation in a combination of simultaneous and sequential modes during a single treatment session.

12. The system of claim 1, wherein the waveform comprises at least one of a sinusoidal waveform, a square waveform, a pulsed waveform, or combinations thereof.

13. The system of claim 1, wherein the machine learning model is further configured to select the waveform based on the physiological data.

14. The system of claim 1, wherein the controller further comprises a safety constraint module configured to limit at least one of energy density, intensity, or temperature of the biological tissue.

15. The system of claim 1, wherein the system is configured to deliver electromagnetic radiation having an energy density ranging from about 1 mJ/cm² to about 1000 J/cm².

16. The system of claim 1, wherein the electromagnetic radiation is configured to perform tissue debridement using electromagnetic radiation wavelengths of at least one of 980 nm, 1470 nm, or combinations thereof.

17. The system of claim 1 configured for at least one of wound healing, pain management, dermatological treatment, deep tissue healing, hair growth stimulation, musculoskeletal therapy, cutting tissue, cropping necrotic tissue, tissue regeneration, or combinations thereof.

18. A method for providing adaptive electromagnetic radiation therapy to a biological tissue of a subject, the method comprising:
emitting electromagnetic radiation from a plurality of electromagnetic radiation sources toward a treatment region of the subject;
acquiring physiological data associated with a state of the treatment region using a plurality of physiological sensors;
processing the physiological data and patient-specific data using a machine learning model to generate a control policy;
and
dynamically controlling at least one of wavelength selection, waveform, temporal sequencing, or intensity of the electromagnetic radiation based on the control policy,
wherein the control policy is iteratively updated during a treatment session based on feedback derived from the physiological data, thereby adaptively regulating electromagnetic radiation delivery to the biological tissue.

19. The method of claim 18, further comprising acquiring baseline physiological state data of the treatment region prior to emitting electromagnetic radiation.

20. The method of claim 18, further comprising acquiring real-time physiological response data during emission of electromagnetic radiation.

21. The method of claim 18, wherein processing the physiological data comprises evaluating a temporal evolution of the treatment region based on baseline data and real-time response data.

22. The method of claim 18, wherein emitting electromagnetic radiation comprises sequentially activating the plurality of electromagnetic radiation sources according to the control policy.

23. The method of claim 18, wherein emitting electromagnetic radiation comprises simultaneously activating at least two electromagnetic radiation sources.

24. The method of claim 18, wherein emitting electromagnetic radiation comprises a combination of simultaneous and sequential activation within a single treatment session.

25. The method of claim 18, further comprising modulating a waveform of the electromagnetic radiation based on output of the machine learning model.

26. The method of claim 18, further comprising applying safety constraints to limit at least one of energy exposure, intensity, or temperature of the biological tissue.

27. The method of claim 18, wherein the machine learning model is updated across multiple treatment sessions based on longitudinal patient response data.

28. The method of claim 18, wherein the method is applied for wound healing, pain management, dermatological treatment, deep tissue healing, hair growth stimulation, musculoskeletal therapy, tissue regeneration, or combinations thereof.
